(19) United States
(12) Patent Application Publication
(10) Pub. No.: US 2012/0296349 A1
(43) Pub. Date: Nov. 22, 2012

(54) Percutaneous Mitral Annulus Mini-Plication

(75) Inventors: Scott R. Smith, Chaska, MN (US); Mark L. Jenson, Greenfield, MN (US)

(73) Assignee: BOSTON SCIENTIFIC SCIMED, INC., Maple Grove, MN (US)

(21) Appl. No.: 13/240,921

(22) Filed: Sep. 22, 2011

Related U.S. Application Data
(60) Provisional application No. 61/487,065, filed on May 17, 2011.

(51) Int. Cl. A61B 17/08; A61B 17/10

(52) U.S. Cl. 606/151

(57) ABSTRACT

A plication clip comprises a first end portion; a second end portion; and a central portion connecting the first end portion to the second end portion. The first end portion is curved toward the second end portion, and the second end portion is curved toward the first end portion. The central portion has a curvilinear profile such that when the clip is deployed, a shorter length between the first end portion and the second end portion is formed. A delivery catheter and methods for deploying the plication clip are also provided.

### CROSS REFERENCE TO RELATED APPLICATIONS

This application claims the benefit of U.S. Provisional Application No. 61/487,065, entitled, “Percutaneous Mitral Annulus Mini-Plication,” by Scott R. Smith and Mark L. Jenson, and filed on May 17, 2011, the entire contents of which being incorporated herein by reference.

### BACKGROUND OF THE INVENTION

The present invention relates generally to devices and methods for the treatment of regurgitation of the mitral valve, which is essentially a check valve located between the left atrium and the left ventricle of the heart. Mitral valve regurgitation occurs when the heart's mitral valve does not close properly. When this occurs, oxygenated blood can flow backwards from the left ventricle into the left atrium during systole, rather than flowing into the aorta and out to the rest of the body. Thus, mitral valve regurgitation may decrease the heart's pumping efficiency, sometimes significantly.

A number of mechanical defects of the mitral valve and the left ventricle may cause mitral valve regurgitation. The valve annulus may be dilated, weakened, or damaged. The valve leaflets may be prolapsed, displaced, or damaged such that they do not close properly. Further, the valve chordae, the papillary muscles, or the left ventricular wall may be damaged or weakened.

Treatment of mitral valve regurgitation often involves surgical procedures to repair the defects or replace the valve. Sutures, surgical clips and staples have previously been used to hold portions of the valve together. While these devices and methods of repairing the valve are effective, they typically require open heart surgery, which is a highly invasive procedure. Previous techniques for the treatment of mitral valve regurgitation are described in U.S. Pat. Nos. 6,676,702; 6,921,407; 6,997,951; 7,635,386; 7,655,015; and US Pat. Pub. No. 2007/0080188, each of which is hereby incorporated by reference in its entirety.

Where the annulus has a mechanical defect, mitral annuloplasty is often performed to make the mitral annulus smaller in the septal-lateral dimension, which allows the mitral valve leaflets to coapt better to reduce mitral valve regurgitation. Mitral annuloplasty typically involves the implantation of a shaped annuloplasty ring, which is loosely sutured inside the left atrium at the mitral annulus. The annuloplasty ring is sized somewhat smaller than the mitral annulus. The suture is tightened to make the annulus smaller, especially in the septal-lateral dimension, or to re-shape the annulus. Using sutures often creates tucks or folds in the tissues in a process.

Percutaneous annuloplasty procedures typically place a rigid structure in the coronary sinus near the location of the mitral annulus. However, these procedures may be less desirable or less effective due to the anatomy of the coronary sinus, the mitral annulus, and the nearby circumflex coronary artery. In particular, by placing a rigid annular cinching device in the coronary sinus, the coronary sinus may cross over the circumflex coronary artery, which may cause compression of the coronary artery.

The present invention provides an apparatus and method for percutaneous plication of a valve assembly that does not require open heart surgery and provides improved cinching results over the prior art.

### BRIEF SUMMARY OF THE INVENTION

The present apparatus for percutaneous plication of a valve assembly (such as the mitral valve annulus in order to reduce valve regurgitation) reduces the annulus by placing at least one plication clip around the valve annulus, each clip shortening the circumference of the annulus.

In at least one embodiment, the plication clip comprises a first end portion; a second end portion; and a central portion connecting the first end portion to the second end portion. In at least one embodiment, the first end portion is curved toward the second end portion, and the second end portion is curved toward the first end portion. The central portion has a curvilinear profile such that when the clip is deployed, the central portion foreshortens the length between the first end portion and the second end portion. In at least one embodiment, the curvilinear profile of the central portion is selected from the group consisting of zig-zags, u-shapes, sinusoidal waves, non-sinusoidal waves, helical coils, reversing helical coils, twists, spirals, planar coils, cochlear-like configurations, and combinations thereof.

In at least one embodiment, an assembly for percutaneus plication of a valve assembly is provided. In at least one embodiment, the assembly comprises at least one plication clip and a delivery device. In at least one embodiment, the delivery device has a proximal end and a distal end. In at least one embodiment, the delivery device comprises at least a retaining mechanism and a shaft. The clip has a loaded state when the clip is loaded onto the delivery device and the clip has a deployed state when the clip is released from the delivery device. When the clip is in the loaded state, the clip has a distance between the first end and the second end. When the clip is in the deployed state, the central portion has a curvilinear profile that shortens the distance between the first end and the second end.

Various exemplary embodiments for plication clips and delivery devices for deploying the plication clips are provided herein.

### BRIEF DESCRIPTION OF THE SEVERAL VIEWS OF THE DRAWING(S)

FIG. 1 is a cross-sectional view of an exemplary human heart.

FIG. 2 is a cross-sectional view of an exemplary human heart, illustrating the heart valves.

FIGS. 3A-3B are top views of one of the valves of the heart showing a reduction in the diameter and inner circumference of the valve annulus when the plication clips of the present invention are used.

FIGS. 4A-4I show embodiments of the plication clip of the present invention.

FIGS. 5A-5B show schematic views of an embodiment of the assembly of the present invention.

FIGS. 6A-6C show schematic views of an embodiment of the assembly of the present invention.

FIGS. 7A-7D show an embodiment of the assembly of the present invention.

FIG. 8 shows an embodiment of the assembly of the present invention.

FIG. 9 shows an embodiment of the assembly of the present invention.

FIG. 10 shows an embodiment of the assembly of the present invention.

FIG. 11 shows an embodiment of the plication clip of the present invention.

FIGS. 12A-12B show an embodiment of the plication clip of the present invention.

FIG. 13 shows an embodiment of the plication clip of the present invention.

### DETAILED DESCRIPTION OF THE INVENTION

While this invention may be embodied in many different forms, there are described in detail herein specific preferred embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

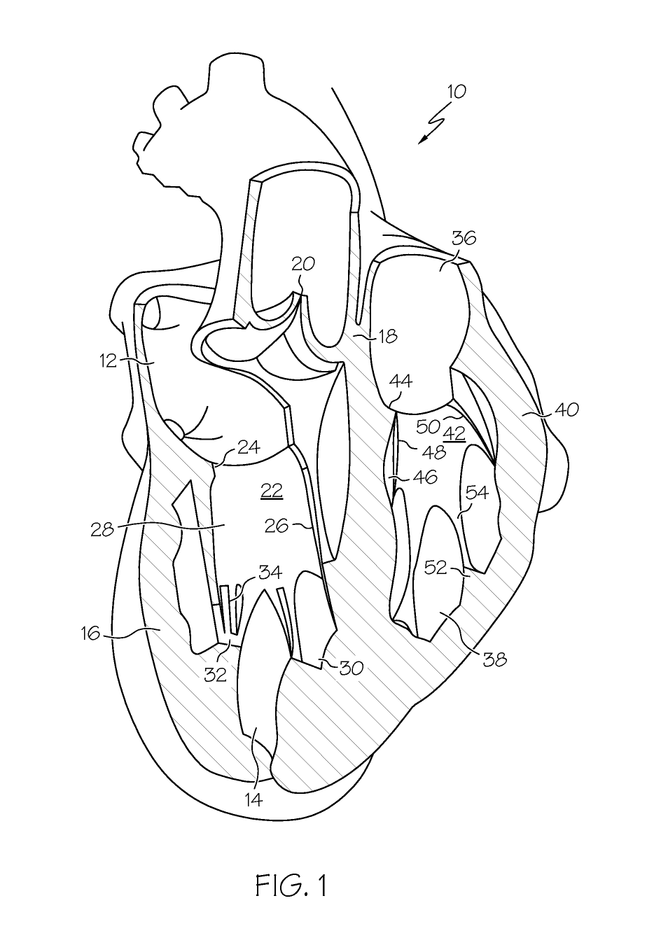

FIG. 1 shows a cross-sectional depiction of a normal human heart 10. The left side of the heart 10 (shown in FIG. 1) includes left atrium 12, left ventricular chamber 14 positioned between left ventricular wall 16 and septum 18, aortic valve 20, and mitral valve assembly 22. The components of the mitral valve assembly 22 include the mitral valve annulus 24, anterior leaflet 26 (sometimes referred to as the aortic leaflet because it is adjacent to the aortic region), posterior leaflet 28, two papillary muscles 30 and 32, and multiple chordae tendineae 34. The papillary muscles 30 and 32 are attached at their bases to the interior surface of the left ventricular wall 16. The chordae tendineae 34 couple the mitral valve leaflets 26 and 28 to the papillary muscles 30 and 32 to support the mitral valve leaflets and to control or restrict leaflet motion.

The right side of the heart 10 (shown in FIG. 1) includes right atrium 36, right ventricular chamber 38 bounded by right ventricular wall 40 and septum 18, and a tricuspid valve assembly 42. The tricuspid valve assembly 42 comprises a valve annulus 44, three leaflets 46, 48, 50, papillary muscles 52 attached to the interior surface of the right ventricular wall 40, and multiple chordae tendinae 54. The chordae tendinae 54 couple the tricuspid valve leaflets 46, 48, 50 to the papillary muscles 52 to support the leaflets 46, 48, 50 and to control or restrict leaflet motion.

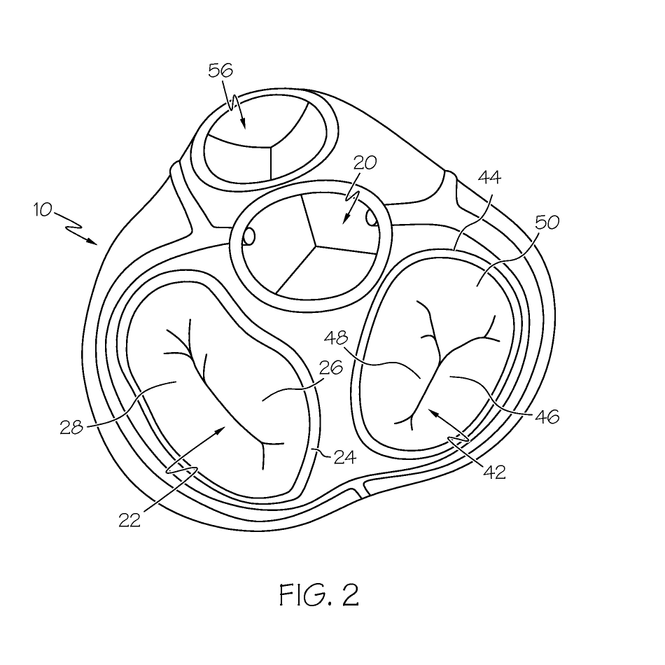

FIG. 2 shows a cross-section of a normal human heart to show a top view of the aortic valve 20, the mitral valve 22, the tricuspid valve 42, and the pulmonary valve 56. As described above, sometimes the valves do not close properly. In the case of the mitral valve, if the valve does not close tightly, blood can flow backward into the heart, thus preventing blood from moving through the body efficiently.

In at least one embodiment, at least one plication clip is used to reduce the circumference of a valve annulus, such as mitral valve annulus 24. Each plication clip cinches the valve annulus in order to shorten the circumference of the annulus to reduce the diameter of the valve opening so that the valve leaflets properly close. At least one plication clip can be used to repair any of the valves shown in FIG. 2 and in other similar tissue structures throughout the body.

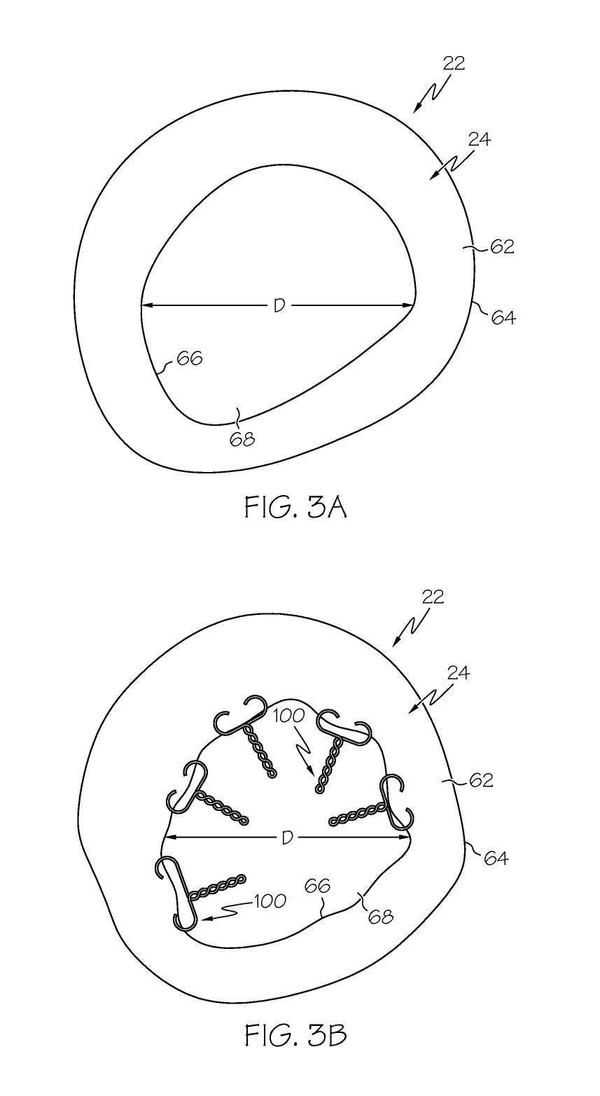

As shown in FIGS. 3A-3B, for example, a valve such as the mitral valve 22 has an outer surface 62 in communication with left atrium 12 (as shown in FIG. 1) and an inner surface (not shown) in communication with the left ventricle 14. The annulus 24 has an outer circumferential surface 64 and an inner circumferential surface 66. The inner circumferential surface 66 defines an opening 68 and has a diameter D.

FIG. 3A shows the annulus 24 prior to insertion of plication clips to reduce the diameter D as desired. The insertion of plication clips into the annulus 24 can reduce the diameter in the septal-lateral dimension, in the commissure-commissure dimension, or both depending on the location of the insertion.

FIG. 3B shows a top view of the mitral valve having multiple plication clips 100 to reduce the annulus 24. Each plication clip 100 is inserted into the annulus, cinching the valve annulus in order to shorten the circumference of the annulus and reduce the diameter. As compared to FIG. 3A, the diameter D is reduced.

In some embodiments, when the clip 100 is inserted into the annulus 24, at least one of the ends of the clip 100 is driven into the inner circumferential surface 66 of the annulus 24 at some distance apart from one another. The central portion 106 of the clip is actuated, deformed, or released to bring the ends of the clip 100 together to shorten the circumference of the annulus 24. In some embodiments, the central portion of the clip 100 is tangent to the circumference of the annulus 24. In other embodiments, the central portion may extend perpendicularly to the surface of the annulus or may be folded over an edge of the annulus.

In some embodiments, the clips 100 are delivered from the atrial side of the heart. In other embodiments, the clips 100 are delivered from the ventricular side of the heart. In some embodiments, the ends of the clip can be driven into the annulus from the atrial side of the heart through the outer surface 62, and in other embodiments, the ends of the clip can be driven into the annulus from the ventricular side of the heart through the inner surface (not shown).

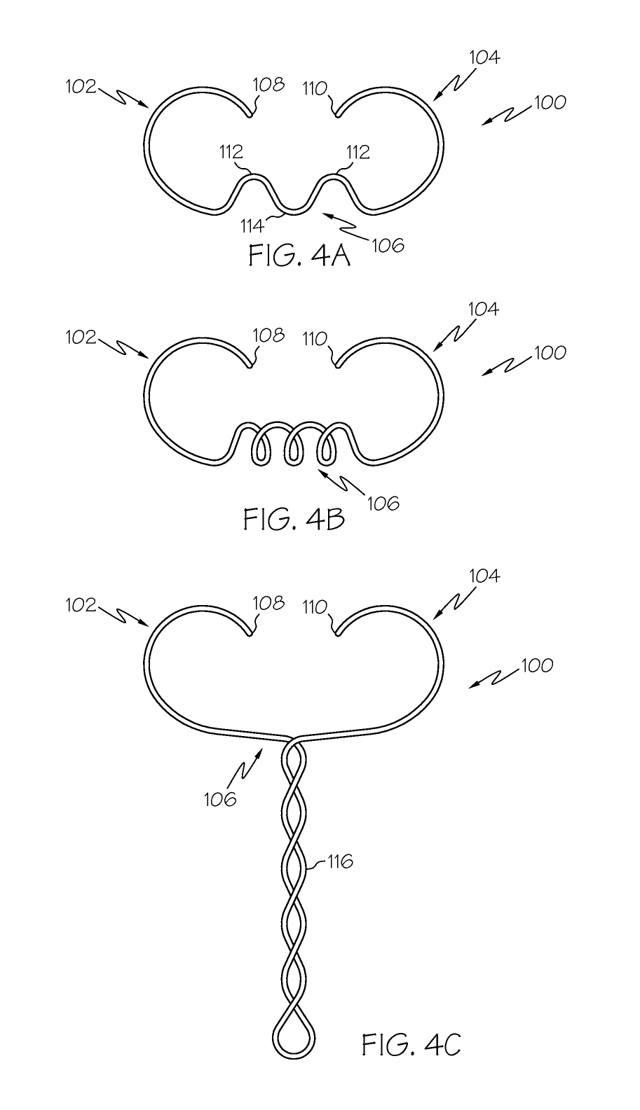

FIGS. 4A-4I show embodiments of the plication clip 100 of the present invention in at least a deployed state. Plication clip 100 has a first end portion 102, a second end portion 104, and a central portion 106 that connects the first end portion 102 to the second end portion 104. In the embodiments shown, the first end portion 102 and the second end portion 104 extend outwardly from the central portion 106. In some embodiments, the plication clip 100 has a generally C-shaped configuration. In some embodiments, such as those shown in FIGS. 4A-4F, the first end portion 102 and the second end portion 104 have curved or hooked members that are bent towards one another. In other embodiments, such as those shown in FIGS. 4G-4I, the first end portion 102 and the second end portion 104 are substantially straight. In some embodiments, the first end portion 102 and the second end portion 104 extend parallel to one another. In at least one embodiment, first end portion 102 and second end portion 104 are co-planar. In other embodiments, first end portion 102 and second end portion 104 are not co-planar.

The first end portion 102 has a free end 108, and the second end portion 104 has a free end 110. In some embodiments, the ends 108, 110 are sharp. In some embodiments, the ends 108, 110 are pointed. In some embodiments, the ends 108, 110 are fitted with tissue anchors, such as barbs (as shown in FIG. 4H). Other types of tissue anchors can be used, such as t-connectors, expanding anchors, treble hooks, suture-based anchoring and other such anchors. During implantation of the clip 100 in the mitral valve, at least the ends 108, 110 are driven into the valve tissue (for example, the annulus tissue or tissue surrounding the annulus) by a delivery device. In some embodiments, the ends 108, 110 function as tissue anchors.

As shown in the exemplary embodiments provided in FIGS. 4A-4I, the central portion 106 can have a variety of curvilinear profiles or configurations that allow the central portion 106 to shorten the length between the end portions 102, 104 (and more particularly, the ends 108, 110). These configurations include, but are not limited to, zig-zags, sinusoidal waves, non-sinusoidal waves, helical coils, reversing helical coils, twists, spirals, planar coils, and combinations thereof.

FIGS. 4A and 4G-4I show the central portion 106 with a sinusoidal wave-like configuration. In the embodiments shown, the central portion 106 has two peaks 112 and one trough 114. In other embodiments, the central portion 106 can have more or less peaks, and more or less troughs.

FIG. 4B shows the central portion 106 having a single helical coil configuration.

FIG. 4C shows the central portion 106 with a twist 116. As shown in FIG. 4C, the twist 116 is positioned in the center of the central portion 106, although in other embodiments, the twist 116 can be offset from the center of the central portion. Multiple twists 116 can also be used.

FIG. 4D shows central portion 106 with a spiral or cochlear-like configuration 118. Although the central portion 106 is shown with only one spiral or cochlear-like configuration 118, multiple spiral or cochlear-like configurations can be used.

FIG. 4E shows the central portion 106 having a first coil portion 120, a second coil portion 122 and a u-shaped portion 124 connecting the first coil portion to the second coil portion. Other configurations are within the scope of the invention.

In some embodiments, such as those shown in FIGS. 4A-4E, the clip 100 comprises a wire having a single thickness along the entire length of the clip. In at least one embodiment, such as the embodiment shown in FIG. 4F, the first end portion 102 and the second end portion 104 each have a first thickness and the central portion 106 has a second thickness that is less than the first thickness. In some embodiments, the thickness of the central portion 106 may be greater than the thickness of the end portions 102, 104. In some embodiments, the first end portion 102 may have a different thickness than the second end portion 104. In some embodiments, the thickness is constant along the respective portion 102, 104, 106. In some embodiments, the thickness tapers towards an end of the respective portion 102, 104, 106. In some embodiments, the thickness varies along the length of the respective portion 102, 104, 106.

In some embodiments, the clip 100 is formed of a wire with a solid, circular cross-section. In other embodiments, the clip 100 is a tubular member having a wall thickness. In some embodiments, the clip 100 has a constant wall thickness. In some embodiments, this wall thickness in the first end portion 102 and the second end portion 104 is different than at least the wall thickness in the central portion. In some embodiments, the first end portion 102 has a different wall thickness than the second end portion 104. In some embodiments, the clip 100 has a non-circular cross-section.

FIGS. 4G-4I show various additional embodiments of clip 100, where the end portions 102, 104 are not curved towards one another, but are either at right angles to the central portion 106 (as shown in FIGS. 4G and 4H) or at an angle 130. In at least one embodiment, angle 130 is between 0 and 90 degrees. In at least one embodiment, the angle between the central portion 106 and the end portion 102 is the same as the angle between the central portion 106 and the end portion 104. In at least one embodiment, the angle between the central portion 106 and the end portion 102 is different than the angle between the central portion 106 and the end portion 104.

In at least one embodiment, clip 100 is formed of a metal, polymer, ceramic or combinations thereof. In some embodiments, clip 100 can comprise layers of different materials.

In some embodiments, the clip 100 is formed of a superelastic material such as a nickel titanium alloy, also known as nitinol, and other superelastic materials with shape memory characteristics. In some embodiments, the clip 100 is formed of a plastically deformable material such as titanium, stainless steel, martensitic nitinol, gold, platinum, elgiloy, mP35N alloy, platinum-enhanced radiopaque stainless steel (PERSS), inconel, and other alloys. In some embodiments, the clip 100 can be formed of an elastic material such as stainless steel and other similar materials. In some embodiments, the material used for the clip 100 can be subjected to different processes such as heat treating, strain hardening, and/or annealing such that the material has certain desired mechanical properties, such as strength, ductility, and elasticity.

In some embodiments, the end portions 102, 104 are formed of a first material and the central portion 106 is formed of a second material that is different than the first material. In some embodiments, the end portion 102 is formed of a material that is different than the material used in end portion 104. In at least one embodiment, the end portions 102, 104 can be stiffer, stronger, or less ductile than the central portion 106. In some embodiments, it can be advantageous to have end portions 102, 104 with a higher strength in order to anchor the clip into the tissue and relatively easier deformation of the central portion 106 in order to shorten the distance between the end portions 102, 104 when delivered in the body.

In some embodiments, the end portions 102, 104 are formed of the same material as the central portion 106, but the end portions 102, 104 can be treated differently than the central portion 106. For example, the central portion 106 can be heat treated while the end portions 102, 104 have not been exposed to any treatment. In some embodiments, the end portions 102, 104 are formed of the same material, but the end portion 102 is treated differently than the end portion 104.

In some embodiments, clip 100 have a coating comprising a non-metallic material, a metallic material, or combinations thereof. In at least one embodiment, the clip 100 has a single coating layer. In at least one embodiment, clip 100 has multiple coating layers.

In at least one embodiment, the clip 100 can have a lubricious coating comprising hydrophilic materials (for example, polyethylene glycol (PEG), polyvinylpyrrolidone (PVP), and polyacrylimide), hydrophobic materials, and combinations thereof.

In at least one embodiment, the clip 100 has a coating layer that comprises a therapeutic agent. The therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

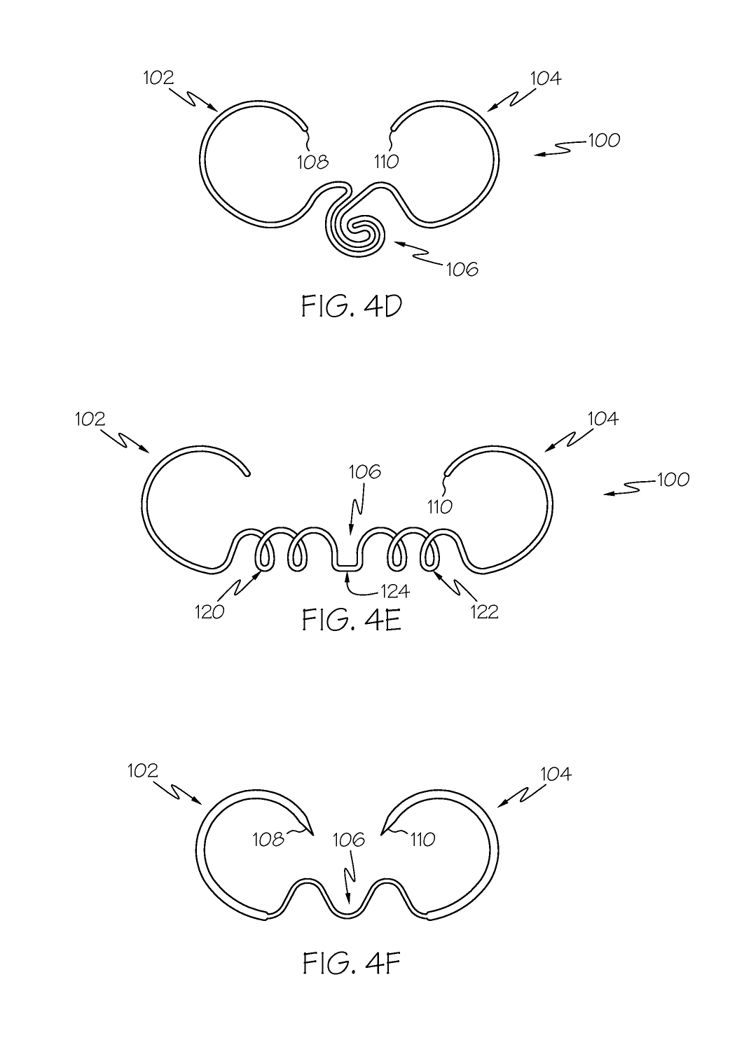

In at least one embodiment, the plication clip 100 is deployed using a delivery device, such as a catheter. FIGS. 5-10 show embodiments of the delivery device for percutaneous plication, which comprises the plication clip 100 and the delivery device. Although the embodiments shown in the figures only have one plication clip 100 on the delivery device, multiple plication clips can be loaded onto the delivery device.

In at least one embodiment, the delivery device is steerable using either a guidewire, a steering sheath, a guide catheter or other like equipment. The delivery device can also be steerable by incorporating a steering mechanism such as one or more tension wires that curve the catheter tip in a desired orientation and support it in that orientation. In other embodiments, the delivery device can be guided and supported by a cage or basket, which can take a form generally like a wire mesh expander, a stent-like braid, a self-expanding stent, or other such framework. One exemplary cage is described in U.S. Provisional Application No. 61/487,053, entitled “Positioning Cage,” and filed on May 17, 2011, hereby incorporated by reference in its entirety. In at least one embodiment, the cage can be formed of a metal or has metal components combined with non-metallic components. In some embodiments, an alignment and support assembly can be used to direct the delivery device as desired and with sufficient backup force to drive the clip 100 into the cardiac tissue.

FIGS. 5A-5B show an embodiment of a delivery device 200 where the plication clip 100 is deployed out of a distal end 201 of the device 200. As shown in the figures, the delivery device 200 comprises a retaining assembly 204 and an inner shaft 206. In at least the embodiment, shown the retaining assembly 204 comprises a pair of jaws 208 with a pair of openings 210. In some embodiments, the retaining assembly 204 is a sheath. In some embodiments, the retaining assembly 204 comprises two halves of a cylinder that define the two openings 210. In at least one embodiment, a pin holds the two halves together and is removed once the clip 100 is ready for full deployment. In some embodiments, the clip 100 is releasably attached to a distal end of the inner shaft 206.

In at least one embodiment, the retaining assembly 204 includes alignment or attachment features such as bends, grooves, indentations, grippers, tabs, slots, stops, detents, tapers, spheres, holes, knobs, and other similar surface features that maintain the clip 100 in a desired position on the delivery device 200. These alignment and attachment features can also be used to form the clip 100 into the desired configuration. In at least one embodiment, the distal end of the shaft 206 includes alignment or attachment features such as bends, grooves, indentations, grippers, tabs, slots, stops, detents, tapers, spheres, holes, knobs, and other similar surface features that maintain the clip 100 in a desired position on the delivery device 200.

In some embodiments, the inner shaft 206 has at least one forming feature, such as cut-aways, sliding covers, sheaths, slots, anvils, driving pins, driving blocks, tabs and other elements to form the central portion 106 of the clip 100 into the desired curvilinear profile. In some embodiments, the retaining assembly 204 has at least one forming feature, such as cut-aways, sliding covers, sheaths, slots, anvils, driving pins, driving blocks, tabs and other elements to form the central portion 106 of the clip 100 into the desired curvilinear profile.

The delivery device 200 can be used with clips comprising elastic, superelastic, and plastically deformable materials.

During deployment of the device, the delivery device 200 can, in some embodiments, be positioned perpendicularly to the valve annulus. In other embodiments, the delivery device 200 is positioned such that it is tangent to the valve annulus.

In some embodiments where the clip 100 comprises a superelastic material, the clip 100 has an initial state where the first end portion is curved toward the second end portion, the second end portion is curved toward the first end portion, the first end is separated from the second end by an initial distance, and the central portion has a curvilinear profile.

In the loaded state shown in FIG. 5A, at least one clip 100 is retained within the retaining assembly 204 and each end of the clip 100 is aligned with a respective opening 210. In at least one embodiment, as shown in FIG. 5A, the central portion 106 of the clip 100 is perpendicular to the longitudinal axis of the delivery device. In other embodiments, the central portion 106 of the clip is parallel to the longitudinal axis of the delivery device. In the loaded state, the clip 100 is elongated and in a substantially U-shaped configuration. The retaining assembly 204 and the inner shaft 206 elongate the clip 100 to straighten out the central portion 106 of the clip. In at least one embodiment, the first end portion 102 is substantially parallel to the second end portion 104 when the plication clip 100 is loaded onto the delivery device. In at least one embodiment, a loaded distance between the first end and the second end is greater than the initial distance.

FIG. 5B shows the clip 100 partially deployed by the device. In at least one embodiment, the inner shaft 206 moves axially relative to the retaining assembly 204. In at least one embodiment, the inner shaft 206 is moved distally to push the end portions 102, 104 of the clip 100 through the openings 210, exposing the end portions 102, 104. In other embodiments, the retaining assembly 204 may be retracted proximally to expose the end portions 102, 104. Where the end portions 102, 104 comprise a superelastic material, the first end portion 102 and the second end portion 104, after being exposed, curve towards one another by recovery of the superelastic material. The end portions 102, 104 are driven into the tissue.

Once the ends are driven into the tissue, the retaining assembly 204 releases the remainder of the plication clip 100 and the distance between the end portions 102, 104 shortens. In some embodiments, the retaining assembly 204 comprises a sheath that is withdrawn to release the plication clip 100. In other embodiments, a button on the device handle can be pressed by the user to release the clip 100 from the retaining assembly 204. The inner shaft 206 and the retaining assembly 204 are retracted, and the clip 100 is fully deployed and the distance between the first end and the second end substantially returns to the initial distance.

Because of its superelastic material properties, the central portion 106 returns substantially to its initial state once it is released, such as shown in FIG. 4A. In this deployed state, the central portion has a curvilinear profile that shortens the distance between the first end 108 and the second end 110. Thus, the clip cinches a portion of the valve annulus in order to shorten the circumference of the annulus and reduce the diameter.

In at least one embodiment, the delivery device 200 shown in FIGS. 5A-5B can be used where at least the central portion 106 of the clip comprises a plastically deformable material. As discussed above, this delivery device 200 has at least one forming feature on the inner shaft 206, the retaining assembly 204, or both. Again, in the loaded state shown in FIG. 5A, each end 108, 110 of the clip 100 is aligned with a respective opening 210. In this loaded state, the clip 100 is elongated and in a substantially U-shaped configuration. During deployment, the inner shaft 206 is moved axially, rotationally or both relative to the retaining assembly 204. The inner shaft 206 is moved axially relative to the retaining assembly 204 in order to expose the end portions 102, 104. Once the end portions 102, 104 are engaged with the tissue of the valve annulus, the forming feature on either the inner shaft 206 or the retaining assembly is actuated by axial movement or rotation to form the curvilinear porile of the clip 100. In some embodiments, the inner shaft 206 is rotated relative to the retaining assembly 204 in order to twist the central portion into the curvilinear profile to cinch a portion of the valve annulus. In some embodiments, the inner shaft 206 is simultaneously moved both axially and rotationally to deploy the delivery device 200.

In at least one embodiment, the device is configured to adjust the depth of insertion into the tissue and the amount of shortening depending on various factors, including the location of deployment.

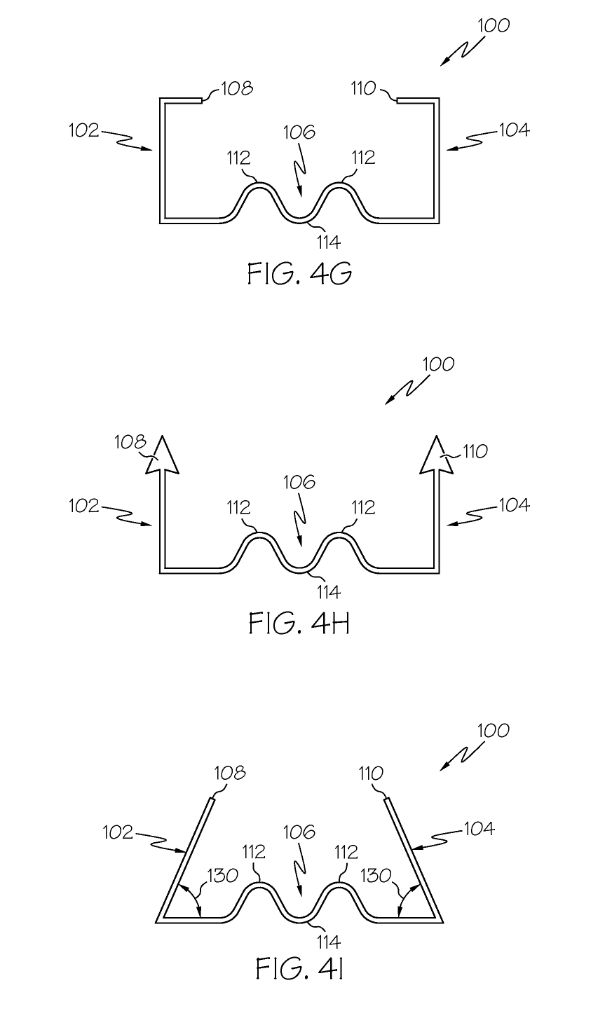

FIGS. 6A-6C schematically show another exemplary delivery device 300 with a retaining device 301 that comprises a first end support structure 302; a second end support structure 304; and a pair of jaws 306. FIG. 6A shows the clip in a loaded state, where the central portion is elongated such that a loaded distance between the first end and the second end is greater than the clip's initial distance. The first end support structure 302 supports and is aligned with the first end portion 102; the second end support structure 304 supports and is aligned with the second end portion 104; and the jaws 306 grip the central portion 106 of the clip. In some embodiments, the first end support structure 302 contacts the first end portion 102, and the second end support structure 304 contacts the second end portion 104 when the clip 100 is loaded onto the delivery device 300. In at least one embodiment, the delivery device 300 has alignment or attachment features such as bends, indentations, grippers, tabs, slots, stops, detents, tapers, spheres, holes, knobs, and other similar surface features that maintain the clip 100 in a centered position on the delivery device 300. The delivery device 300 holds the clip 100 in a low-profile delivery position and allows or produces the deformation of the clip during deployment.

During deployment of the catheter, as shown in FIG. 6B, the jaws 306 are moved closer together to straighten the central portion such that the end portions 102, 104 extend outwardly from the delivery device 300. Where the end portions 102, 104 are comprised of a superelastic material, the first end portion 102 and the second end portion 104, after being exposed, curve towards one another by recovery of the superelastic material. In some embodiments, the straightening of the central portion can increase the length of the clip 100, which is beneficial to driving the clip 100 into the tissue without prematurely shortening the clip 100.

As shown in FIG. 6C, after the end portions 102, 104 of the clip 100 are anchored into the tissue, the jaws 306 open and the central portion 106 is released by actuation of a release mechanism. For example, the jaws 306 can be opened by pushing an actuator or by releasing tension on a spring associated with the jaws 306. When the clip is deployed, the central portion 106 changes shape to return substantially to its initial state such that the distance between the end portions 102, 104 is shortened to cinch the valve tissue. Importantly, the clip 100 is sufficiently anchored prior to the cinching, thus lowering the chance of dislodgment of the anchors during cinching. In some embodiments, the support structures 302, 304 can move inward in order to encourage cinching and to help keep the curved ends 102, 104 anchored in the cardiac tissue during cinching.

Although the above describes use of the delivery device 300 with a clip 100 that comprises a superelastic material, the delivery device 300 may also be used with a clip that comprises a plastically deformable material. In some embodiments, the delivery device has at least one forming feature such as openings, cut-aways, sliding covers, sheaths, slots, anvils, driving pins, driving blocks, tabs and other elements to form the central portion 106 of the clip 100 into the desired curvilinear profile. In some embodiments, the end support structures 302, 304 also have anvils and other elements to curve the end portions towards one another. For example, end portions 102, 104 can be curved by using an anvil-like support structure 302, 304 to bend the end portions 102, 104 into the desired shape. After the end portions 102, 104 of the clip 100 are bent or curved into the desired shape and anchored into the tissue, the jaws 306 of the catheter have forming pins, anvils, or other structures. When actuated, these structures apply forces to bend the central portion of the clip 100 into a curved, zigzag, twisted or other curvilinear configuration (such as the configurations shown in FIGS. 4A-4E), thus shortening the distance between the end portions 102, 104 and cinching the tissue once the clip 100 is deployed. In at least one embodiment, a secondary device separate from the delivery device 300 can be provided to deform the central portion of a plastically deformable clip into the desired curvilinear profile.

During deployment of the device, the delivery device 300 can, in some embodiments, be positioned perpendicularly to the valve annulus. In other embodiments, the delivery device 300 is positioned such that it is tangent to the valve annulus.

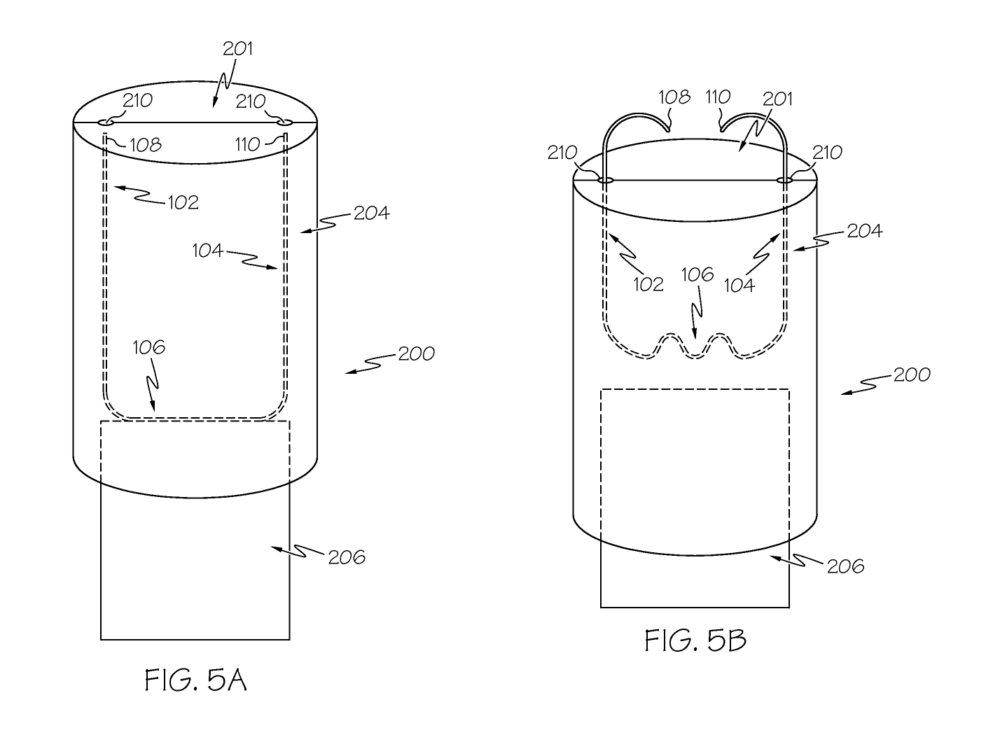

FIG. 7A-7D show another example of a delivery device for plication clip. Here, the delivery device 400 has a proximal end 402, a distal end 404, a shaft 412 with a retaining mechanism 414, and a sheath 416 concentrically positioned about the shaft 412. In some embodiments, the delivery device 400 has a pull wire (not shown). The central portion 106 of the clip is retained towards a distal end 419 of the shaft 412 by the retaining mechanism 414, and in some embodiments, the clip is retained with the pull wire. In at least one embodiment, the retaining mechanism 414 comprises a round anvil, such as a pin 420, that is driven through the shaft 412 perpendicularly to the longitudinal axis of the shaft 412.

As shown in FIG. 7A, in the loaded state, the clip 100 is wrapped about the pin 420 and retained within sheath 416. In some embodiments, the end portions 102, 104 overlap when loaded. In some embodiments, the central portion 106 of the clip 100 is engaged with the pull wire.

During deployment, as shown in FIG. 7B, the clip is pushed out of the distal end 404 of the delivery device by axial movement of the shaft 412, releasing the end portions 102, 104. Where the end portions comprise a superelastic material, the first end portion 102 and the second end portion 104, after being exposed, curve towards one another by recovery of the superelastic material. The end portions 102, 104 are driven into the tissue. In at least one embodiment, the end portions 102, 104 engage with the annulus by pushing the entire delivery device 400 distally against the annulus. In at least one embodiment, the sheath 416 has slots 422 at a distal end of the sheath 416, and the sheath can be rotated until the slots 422 align with the end portions 102, 104.

Where the central portion comprises a superelastic material, once the end portions have been driven into the tissue, the clip is released such as by actuation of the pull wire or a release mechanism associated with the pin 420, and the central portion 106 returns substantially to its initial state, such as is shown in FIG. 3A, to cinch a portion of the valve annulus in order to shorten the circumference of the annulus and reduce the diameter.

Where the central portion comprises a plastically deformable material, the shaft 412 is retracted once the end portions have been driven into the tissue, as shown in FIG. 7C. The shaft 412 is rotated relative to the sheath 416 to twist the central portion 106 of the clip 100. The sheath 416 is then fully retracted as shown in FIG. 7D and the clip is released such as by actuation of the pull wire or a release mechanism associated with the pin 420.

In some embodiments where the end portions comprise a plastically deformable material, the device 400 further comprises a forming feature such as openings, cut-aways, sliding covers, sheaths, slots, anvils, driving pins, driving blocks, tabs and other elements. In at least one embodiment, the forming feature is an anvil. In the loaded state, the end portions 102, 104 are wrapped around the anvil to curve the ends. In some embodiments, the end portions 102, 104 overlap when loaded. In at least one embodiment, the anvil comprises at least two components and the clip is advanced into the anvil, deforming the clip into the desired shape. In at least one embodiment, the forming feature is engaged with the shaft 412. In at least one embodiment, the forming feature is engaged with the sheath 416.

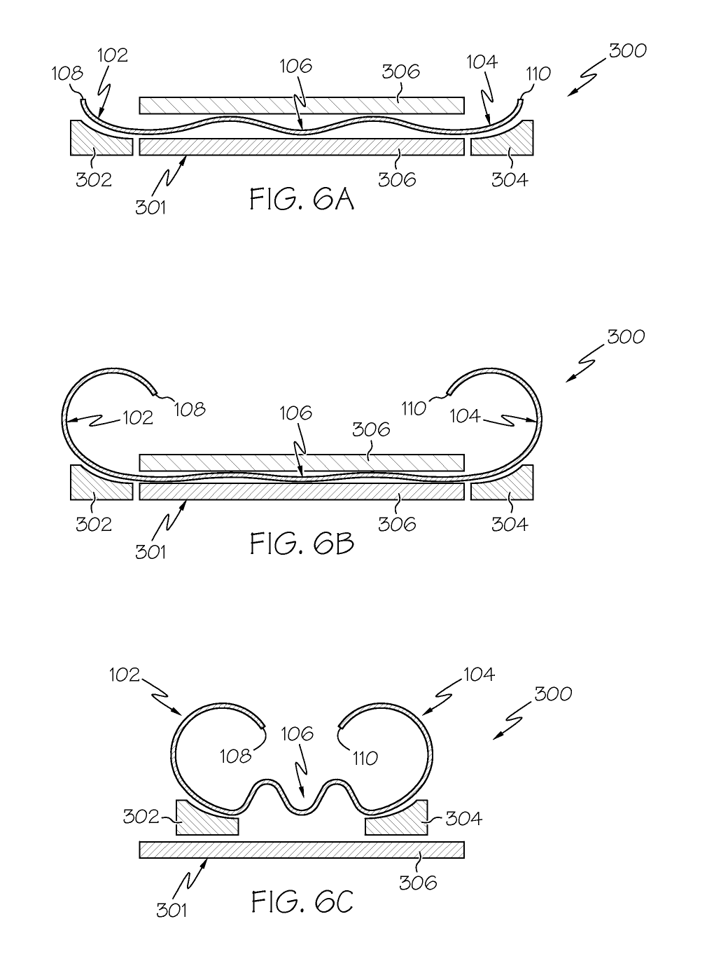

FIG. 8 shows another example of a delivery device for plication clip 100. Here, the delivery device 500 has an outer sheath 502, and inner sheath 503 and a retaining mechanism 504, which comprises an inner shaft 506 that has a tab feature 508. In some embodiments, the retaining mechanism 504 also has at least one gripping member 510.

When loaded onto the delivery device 500, the clip is elongated and held within the retaining mechanism 504 by at least the tab feature 508. In at least one embodiment, the end portions 102, 104 of the clip 100 are held by a groove 512 in each gripping member 510. In at least one embodiment, the central portion 106 of the clip 100 is engaged with the tab feature 508. In other embodiments, the central portion 106 is not engaged with the tab feature 508. During deployment, the outer sheath 502 is retracted, exposing the end portions 102, 104, which are driven into the tissue. The inner shaft 506 is rotated, causing tab feature 508 to rotate, which deforms the central portion 106 to bring end portions 102, 104 closer together. In at least one embodiment, the inner sheath 503 has slots 522 at either end of the sheath. The slots 522 each extend inwardly from an end of the sheath. The inner shaft 506 is rotated until the end portions contact the innermost surface 523 of the slot. Once the inner shaft 506 has been fully rotated and the distance between the end portions 102, 104 has been adequately shortened, at least the inner sheath 503 is separated from at least a portion of the retaining mechanism 504. At least the inner sheath 503 remains at the delivery location with the clip 100 when fully deployed.

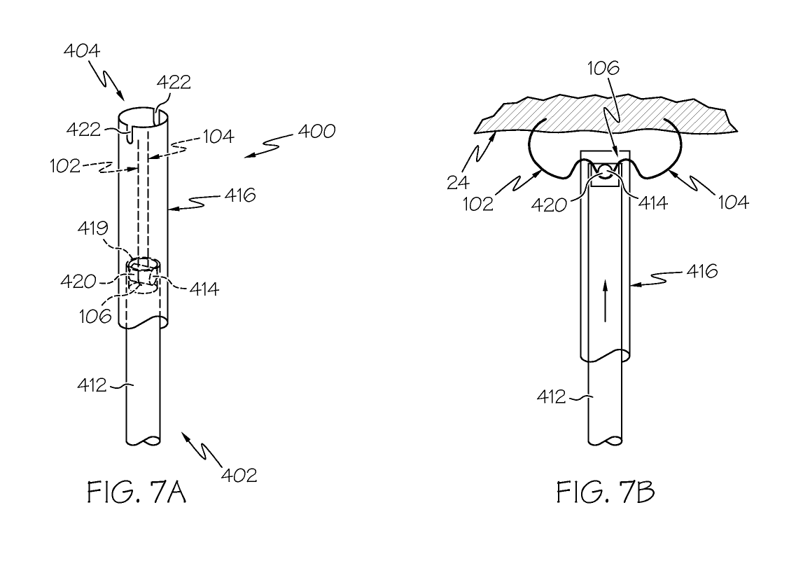

FIG. 9 shows a delivery device 600 for use with the clip 100. Delivery device 600 comprises an outer sheath (not shown), an inner sheath 608, a shaft 610 disposed within the inner sheath 608, and a pull wire (not shown). In some embodiments, the shaft 610 has a retaining mechanism 612 that allows the first end portion 102 and the second end portion 104 to be properly aligned on the shaft 610. The retaining mechanism 612 can also be connected to the pull-wire such that actuation of the pull-wire releases the clip from the delivery device 600. In at least one embodiment, the retaining mechanism 612 comprises a tab.

When loaded onto the device, the clip 100 is elongated in some embodiments such that a loaded distance between the first end and the second end of the clip 100 is greater than the deployed distance. Once the delivery device 600 reaches the location on the valve annulus for deployment, the outer sheath can be withdrawn to expose the end portions 102, 104. Where at least the end portions 102, 104 comprise a superelastic material, the end portions 102, 104 curve towards one another by recovery of the superelastic material. Where the end portions 102, 104 comprise a plastically deformable material, in some embodiments, the outer sheath, the shaft 610, or both have cut-aways, sliding covers or sheaths, slots, anvils, driving pins or blocks, tabs and other elements to form the end portions 102, 104.

The exposed hooked sections 102, 104 are driven into the tissue and the central portion 106 is shortened to short the distance between the end portions 102, 104. In at least the embodiment shown in FIG. 9, central portion 106 comprises a coil disposed about shaft 610. In at least one embodiment, the central portion 106 is engaged with the retaining mechanism 612, which is shown in FIG. 9 as a tab. The shaft 610 is rotated relative to the inner sheath 608 to shorten the central portion 106, and thus shorten the distance between the first end portion 102 and the second end portion 104. In at least one embodiment, actuation of the retaining mechanism 612 releases the shaft 610 from the clip 100. In some embodiments, the entire delivery device 600 is retracted and removed from the vasculature. In at least one embodiment, the inner sheath 608 remains about the clip 100 after the clip 100 is fully deployed.

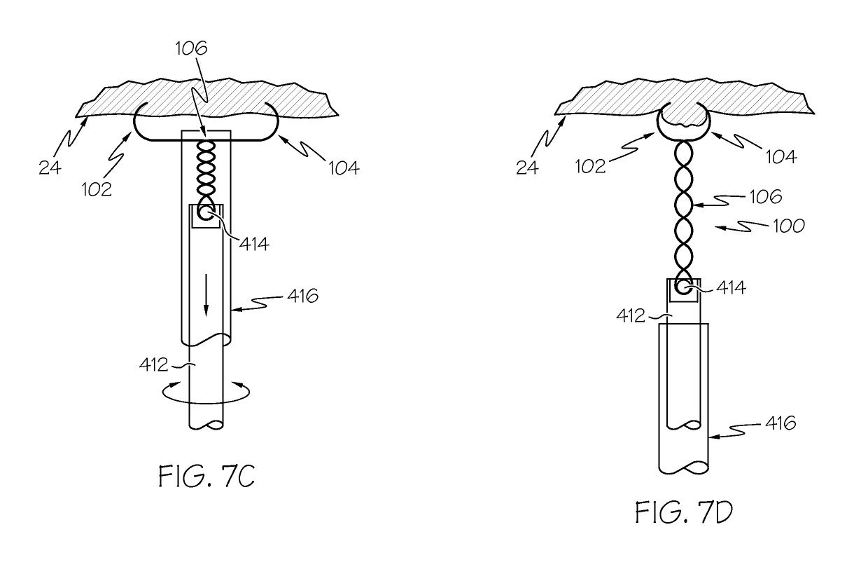

FIG. 10 is a modification to the delivery devices described above that enables any of the tangentially applied clips described herein to be delivered from a perpendicular approach, or vice versa. A member 702 is pivotally engaged with the delivery device 700 that holds the plication clip 100. When the member 702 is at the desired location, the user can rotate the delivery device 700 relative to the member 702 to deploy the clip at any desirable angle.

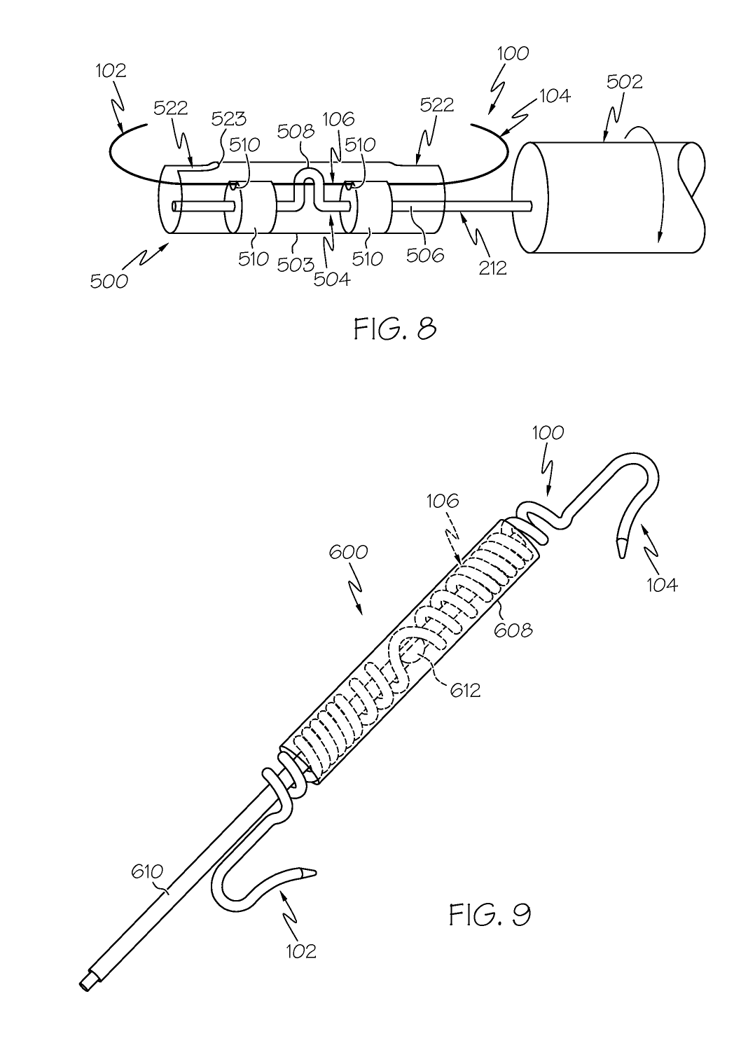

In at least one embodiment, the clip can comprise two separate components. FIG. 11 shows a clip 900 having a first end portion 902 and a second end portion 904. The first end portion 902 and the second end portion 904, which are separate. First end portion 902 has a hooked section 908 defining the first end of the clip and a spiraled section 910. Second end portion 904 has a hooked section 912 defining the second end of the clip and a spiraled section 914. In at least one embodiment, the spiraled section 914 of the second end portion 904 has a helix that is opposite to a helix of the spiraled section 910 of the first end portion 902. When the clip 900 is deployed with a delivery device, the spiraled section 914 of the second end portion 904 engages with the spiraled section 910 of the first end portion 902 to shorten the distance between the first end and the second end. As such, the spiraled section 914 and the spiraled section 910 are interconnected, shortening the distance between the first end portion 902 and the second end portion 904.

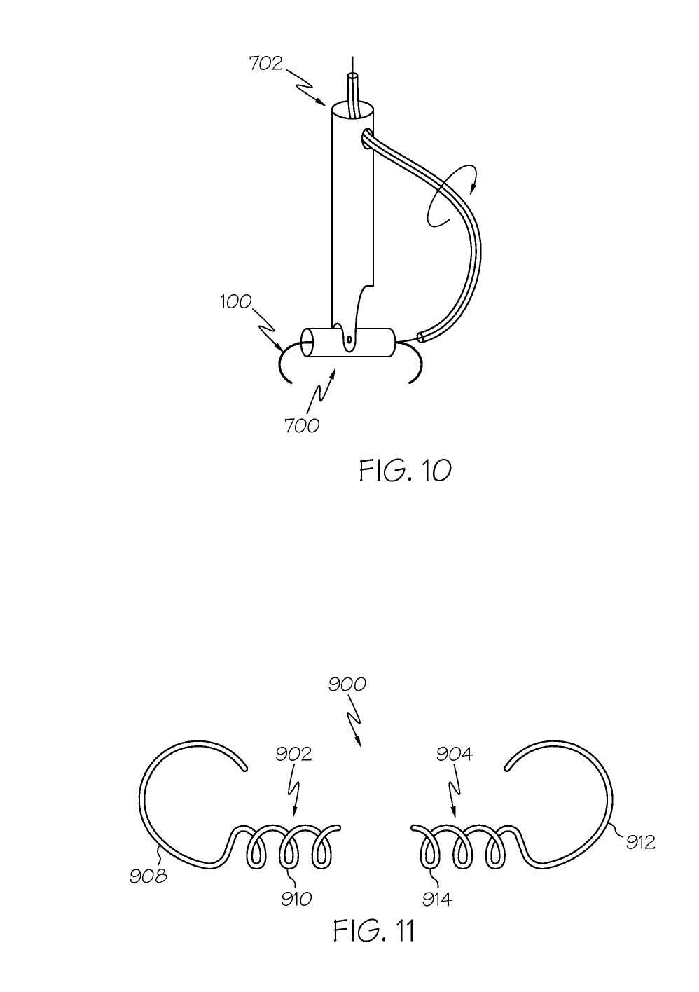

FIGS. 12A-12B show another embodiment of the clip 950. In this embodiment, the clip 950 has end portions 952, 954 and central portion 956. First end portion 952 has a first section 958 and a second section 960. Second end portion 954 has a first section 962 and a second section 964. The second sections 960, 964 both have outer threads 966, 968. Central portion 956 is a tubular member with an inner thread 970 that mates with the outer threads 966, 968 on each of the second sections 960, 964 of the end portions 952, 954. At least when deployed, the first section 958 of the first end portion 952 is curved towards the first section 962 of the second end portion 954, and vice versa.

In this embodiment, torsional or rotational movement of the central portion 956 in a first direction shortens the distance between the first section 958 of the first end portion 952 and the first section 962 of the second end portion 954 to cinch the tissue. Torsional or rotational movement of the central portion 956 in a second direction increases the distance between the first end portion 952 and the second end portion 954. In at least one embodiment, the central portion 956 is rotated until the end of the second section 964 of the second end portion 954 contacts the end of the second section 960 of the first end portion 952.

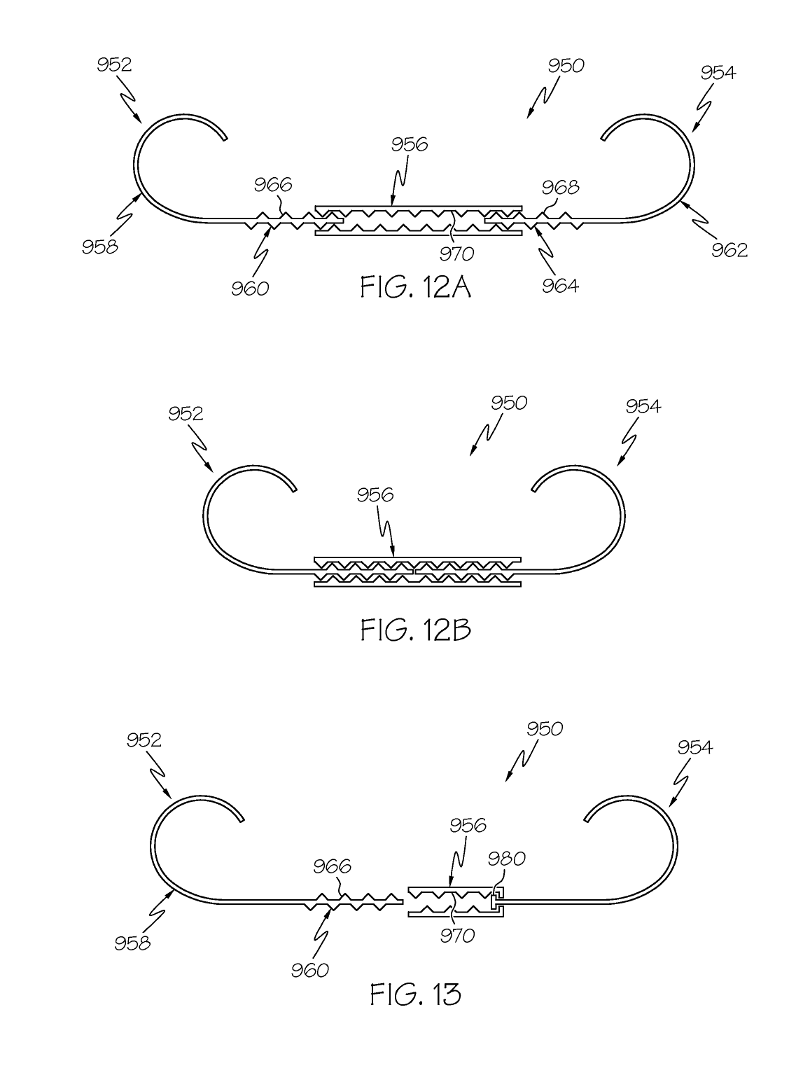

FIG. 13 shows another embodiment of the clip 950 where only the first end portion 952 has a second section 960 with outer threads 966 that mate with the inner threads 970 of the central portion 956. Again, torsional or rotational movement of the central portion 956 in a first direction shortens the distance between the first end portion 952 and the second end portion 954 to cinch the tissue. In at least one embodiment, the second end portion 954 has a knob 980 that prevents axial movement of the second end portion 954 relative to the central portion 956, while allowing rotational movement of the central portion 956.

To deliver the devices shown in FIGS. 12A-12B and 13, a delivery device holds the end portions 952, 954 straight when loaded onto the device. Once the device reaches the delivery location, a sleeve or sheath is retracted which allows the end portions 952, 954 to be exposed and partially curved. The end portions 952, 954 are then driven into the tissue. The delivery device rotates the central portion 956 to draw the end portions 952, 954 together. The end portions 952, 954 and the central portion 956 must then be secured to each other, either through an interference fit, a locking or ratcheting mechanism, or through a forming process so that the various components are not loosened. In an alternative embodiment, a split threaded sleeve can be used to retain the end portions 952, 954, and rotational movement by the delivery device threads the sleeve together to expose the end portions 952, 954. This same rotational movement can rotate the central portion 956 to pull the end portions 952, 954 together. In any embodiment of the delivery device, various gears, teeth, levers, pins, shafts, threads, latches, ratchets, rollers, lumens, locks, and other components can be used. Adhesives, fusible links, and various attachment and separation means can also be employed.

In at least one embodiment, multiple clips 100 can be loaded onto any of the delivery devices described herein. Any of the exemplary delivery devices described in this application can be modified in accordance with the device shown in FIG. 10 to deploy the clips tangentially to the annulus, rather than perpendicular to the annulus.

Although the device and method as described above references deploying a plication clip at the mitral valve, it is within the scope of this invention that the plication clip can be used to close other valves and other bodily tissue structures.

Although particular features are shown or described with respect to particular embodiments disclosed herein, these features can be combined with the features or substituted for the features of other embodiments.

In addition, U.S. Provisional Application No. 61/487,083 entitled “Annuloplasty Ring with Anchors Fixed by Curing Polymer,” and filed on May 17, 2011; U.S. Provisional Application No. 61/487,053, entitled “Positioning Cage,” and filed on May 17, 2011; U.S. Provisional Application No. 61/487,063, entitled “Corkscrew Annuloplasty Device,” and filed on May 17, 2011; and U.S. Provisional Application No. 61/487,072 “Annuloplasty Ring with Piercing Wire and Segmented Wire Lumen,” and filed on May 17, 2011, are each hereby incorporated by reference in their entireties.

In at least one embodiment, a plication clip comprises a first end portion that defines a first end; a second end portion that defines a second end; and a central portion connecting the first end portion to the second end portion; wherein the first end portion is curved toward the second end portion, the second end portion is curved toward the first end portion, and the central portion has a curvilinear profile. In one embodiment, at least the central portion comprises a material selected from the group consisting of elastic materials, superelastic materials and plastically deformable materials. In one embodiment, the first end portion, the second end portion, and the central portion are each formed of the same material. In one embodiment, the first end portion and the second end portion are formed of a first material and the central portion is formed of a second material, wherein the second material is different from the first material. In one embodiment, the first end portion and the second end portion have a first width and the central portion has a second width, wherein the second width is different from the first width. In one embodiment, the curvilinear profile of the central portion is selected from the group consisting of zig-zags, u-shapes, sinusoidal waves, non-sinusoidal waves, helical coils, reversing helical coils, twists, spirals, planar coils, cochlear-like configurations, and combinations thereof. In one embodiment, the first end portion, the second end portion, and the central portion have a constant thickness. In one embodiment, the first end portion has a first thickness, the second end portion has a second thickness and the central portion has a third thickness, wherein the third thickness is different from at least one of the first thickness and the second thickness.

In at least one embodiment, a plication clip comprises a first end portion that defines a first end; a second end portion that defines a second end; and a central portion connecting the first end portion to the second end portion; wherein the plication clip has a loaded state and a deployed state; wherein in the loaded state, the first end is separated from the second end by a distance; wherein in the deployed state, the first end portion is curved toward the second end portion, the second end portion is curved toward the first end portion, and the central portion has a curvilinear profile that shortens the distance between the first end and the second end. The central portion can comprise a material selected from the group consisting of elastic materials, superelastic materials and plastically deformable materials. In one embodiment, the first end portion, the second end portion, and the central portion are each formed of the same material. In one embodiment, the first end portion and the second end portion are formed of a first material and the central portion is formed of a second material, wherein the second material is different from the first material. In one embodiment, the curvilinear profile of the central portion is selected from the group consisting of zig-zags, u-shapes, sinusoidal waves, non-sinusoidal waves, helical coils, reversing helical coils, twists, spirals, planar coils, cochlear-like configurations, and combinations thereof.

In at least one embodiment, the plication clip comprises a first end portion that defines a first end; a second end portion that defines a second end; and a central portion connecting the first end portion to the second end portion; wherein the plication clip has an initial state, a loaded state and a deployed state; wherein in the initial state, the first end portion is curved toward the second end portion, the second end portion is curved toward the first end portion, the first end is separated from the second end by an initial distance, and the central portion has a curvilinear profile; wherein in the loaded state, at least the central portion is elongated such that a loaded distance between the first end and the second end is greater than the initial distance; wherein in the deployed state, a distance between the first end and the second end is substantially equal to the initial distance. The central portion can comprise a superelastic material. In one embodiment, the curvilinear profile of the central portion is selected from the group consisting of zig-zags, u-shapes, sinusoidal waves, non-sinusoidal waves, helical coils, reversing helical coils, twists, spirals, planar coils, cochlear-like configurations, and combinations thereof.

In at least one embodiment, the plication clip comprises a first end portion comprising a first section that defines a first end and a second section with outer threads; a second end portion comprising a first section that defines a second end; and a central portion connecting the first end portion to the second end portion, the central portion comprising a tubular member with inner threads that mate with the outer threads of the first end portion, wherein rotational movement of the central portion in a first direction shortens a distance between the first and the second end. In one embodiment, the second end portion further comprises a second section with outer threads that mate with the inner threads of the central portion.

While other embodiments are also described above, in at least one embodiment an assembly for percutaneous plication of a valve assembly comprises a clip and a delivery device. The clip comprises a first portion comprising a first hooked section and a first spiraled section, the first hooked section defining a first end of the clip, and a second portion comprising a second hooked section and a second spiraled section, the second hooked section defining a second end of the clip, wherein the second portion is separate from the first portion. The delivery device comprises an inner sheath, a shaft disposed within the inner sheath, and a release mechanism. When the clip is loaded onto the delivery device, the first portion and the second portion are disposed about the shaft; and the first end and the second end are separated by a loaded distance. When the clip is deployed, the first spiraled section is engaged with the second spiraled section, and a distance between the first and the second end is shorter than the loaded distance. In one embodiment, when the clip is loaded onto the delivery device, the first portion and the second portion are disposed about the shaft; and during deployment, the shaft is rotated to engage the first spiraled section with the second spiraled section. In at least one embodiment, the first spiraled section has a helix in a direction opposite to of a helix of the second spiraled section.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to.” Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims.

This completes the description of the preferred and alternate embodiments of the invention. Those skilled in the art may recognize other equivalents to the specific embodiment described herein which equivalents are intended to be encompassed by the claims attached hereto.

1. An assembly for percutaneous plication of a valve assembly, the assembly comprising:
a clip, the clip comprising a first end portion defining a first end; a second end portion defining a second end; and a central portion connecting the first end portion to the second end portion; and
a delivery device, the delivery device having a proximal end and a distal end, the delivery device comprising a retaining mechanism and a shaft;
wherein the clip has a loaded state when the clip is loaded onto the delivery device and the clip has a deployed state when the clip is released from the delivery device;
wherein in the loaded state, the clip has a distance between the first end and the second end,
wherein in the deployed state, the central portion has a curvilinear profile that shortens the distance between the first end and the second end.

2. The assembly of claim 1, wherein in the deployed state, the first end portion is curved toward the second end portion, the second end portion is curved toward the first end portion.

3. The assembly of claim 1, wherein the curvilinear profile of the central portion is selected from the group consisting of zig-zags, u-shapes, sinusoidal waves, non-sinusoidal waves, helical coils, reversing helical coils, twists, spirals, planar coils, cochlear-like configurations, and combinations thereof.

4. The assembly of claim 1, wherein at least the central portion comprises a material selected from the group consisting of elastic materials, superelastic materials and plastically deformable materials.

5. The assembly of claim 1, wherein the retaining mechanism comprises a pair of jaws.

6. The assembly of claim 5, wherein the retaining mechanism further comprises a first end support structure and a second end support structure.

7. The assembly of claim 6, wherein when the clip is loaded onto the delivery device, the first end portion is aligned with the first end support structure, the second end portion is aligned with the second end support structure, and the central portion is held between the pair of jaws.

8. The assembly of claim 5, wherein the jaws define openings.

9. The assembly of claim 8, wherein when the clip is loaded onto the delivery device, each end of the clip is aligned with one of the openings.

10. The assembly of claim 1, wherein the delivery device further comprises at least one alignment feature.

11. The assembly of claim 1, wherein the delivery device further comprises at least one attachment feature.

12. The assembly of claim 1, wherein the delivery device further comprises at least one forming member.

13. The assembly of claim 12, wherein the at least one forming member is selected from the group consisting of cut-aways, sliding covers, sheaths, slots, anvils, driving pins, blocks, tabs, and combinations thereof.

14. The assembly of claim 13, wherein the at least one forming member bends the central portion into the curvilinear profile during deployment.

15. The assembly of claim 1, wherein the clip is loaded onto the first end of the delivery device such that the central portion of the clip is perpendicular to a longitudinal axis of the delivery device.

16. The assembly of claim 1, wherein the clip is loaded onto an outer surface of the delivery device such that the central portion of the clip is parallel to a longitudinal axis of the delivery device.

17. A method for percutaneous plication of a tissue comprising:
loading a clip on a delivery device, the clip comprising a first end portion having a first free end; a second end portion having a second free end; and a central portion connecting the first end portion to the second end portion, the delivery device having a proximal end and a distal end, the delivery device comprising a retaining mechanism and a shaft;
driving the first free end and the second free end into the tissue;
releasing the central portion of the clip by actuating the retaining mechanism to fully deploy the clip.

18. The method of claim 17, wherein when the clip is loaded onto the delivery device, the first free end is separated from the second free end by a loaded distance, and when the delivery device is deployed, a deployed distance between the first free end and the second free end is shorter than the loaded distance.

19. The method of claim 17, wherein at least the central portion of the clip is formed of a material selected from the group consisting of elastic materials, superelastic materials and plastically deformable materials.
